(19) United States
(12) Patent Application Publication
(10) Pub. No.: US 2024/0021275 A1
(43) Pub. Date: Jan. 18, 2024

(54) MACHINE-LEARNED MODELS FOR SENSORY PROPERTY PREDICTION

(71) Applicant: Osmo Labs, PBC, Somerville, MA (US)

(72) Inventors: Alexander Wiltschko, Somerville, MA (US); Wei Qian, Urbana, IL (US); Jennifer Wei, Somerville, MA (US); Benjamin Manuel Sanchez-Lengeling, Somerville, MA (US); Brian Kihoon Lee, Somerville, MA (US); Yiliu Wang, Los Altos, CA (US)

(73) Assignee: Osmo Labs, PBC, Somerville, MA (US)

(21) Appl. No.: 18/036,707

(22) PCT Filed: Nov. 12, 2021
(86) PCT No.: PCT/US21/59078

§ 371 Date: May 12, 2023

Related U.S. Application Data
(60) Provisional application No. 63/113,256, filed on Nov. 13, 2020.

(51) Int. Cl. G16C 20/30; G16C 20/70; G06N 3/04; G06N 3/096

(52) U.S. Cl. CPC G16C 20/30; G16C 20/70; G06N 3/04; G06N 3/096

(57) ABSTRACT

A computer-implemented method for predicting whether a molecule will be a good mosquito repellent is disclosed. The method includes obtaining a machine-learned prediction model obtained by transfer learning. The model has been trained using a first, larger training dataset for an odour prediction task and with a second, smaller training dataset for predicting whether a molecule would function as a mosquito repellent. The method further includes obtaining input data that describes a chemical structure of a selected molecule, providing the input data that describes the chemical structure of the selected molecule as input to the machine-learned prediction model, receiving prediction data descriptive of whether the selected molecule would be a good mosquito repellent as an output of the machine-learned sensory prediction model and providing the prediction data as output.

### RELATED APPLICATIONS

This application claims priority to and the benefit of U.S. Provisional Patent Application No. 63/113,256, filed Nov. 13, 2020. U.S. Provisional Patent Application No. 63/113,256 is hereby incorporated by reference in its entirety.

### FIELD

The present disclosure relates generally to machine-learned models for sensory property prediction. More particularly, the present disclosure relates to machine-learned models that are first trained for a first sensory prediction task and used to make predictions for a second sensory prediction task.

### BACKGROUND

The relationship between a molecule's structure and its olfactory perceptual properties (e.g., the perceived scent of a molecule) is complex, and, to date, generally little is known about such relationships. For example, the flavor and fragrance industries generally rely on trial-and-error, heuristics, and/or mining natural products to provide commercially useful products having desired sensory properties (e.g., olfactory properties). There is generally a lack of meaningful principles for organizing the olfactory environment, though it is known that mapping between molecular structure and scent can be very nonlinear, such that small changes in molecules can yield large changes in olfactory quality. Additionally, the inverse can also be true, where diverse families of molecules may exhibit similar olfactory characteristics.

### SUMMARY

Aspects and advantages of embodiments of the present disclosure will be set forth in part in the following description, or can be learned from the description, or can be learned through practice of the embodiments.

One example aspect of the present disclosure is directed to a computer-implemented method for training a sensory prediction model for predicting sensory properties for a prediction task having limited available training data for a second sensory prediction task. The method can include obtaining, by a computing system comprising one or more computing devices, a first sensory prediction task training dataset including first training data associated with a first sensory prediction task, the first training data comprising molecular structure data labeled with first sensory properties associated with the first sensory prediction task. The method can include training, by the computing system, a machine-learned sensory prediction model based at least in part on the first sensory prediction task training dataset to predict the first sensory properties associated with the first sensory prediction task. The method can include obtaining, by the computing system, a second sensory prediction task training dataset including second training data associated with a second sensory prediction task, the second training data including molecular structure data labeled with second sensory properties associated with the second sensory prediction task, wherein a number of data items of the first sensory prediction task training dataset is greater than a number of data items of the second sensory prediction task training dataset. The method can include training, by the computing system, the machine-learned sensory prediction model based at least in part on the second sensory prediction task training dataset to predict the second sensory properties associated with the second sensory prediction task.

Another example aspect of the present disclosure is directed to a computer-implemented method for predicting sensory properties for a prediction task having limited available training data. The method can include obtaining, by one or more computing devices, a machine-learned sensory prediction model trained to predict sensory properties of molecules based at least in part on chemical structure data associated with the molecules, wherein the machine-learned sensory prediction model is trained using a first sensory prediction task training dataset for a first sensory prediction task. The method can include obtaining, by the one or more computing devices, input data that describes a chemical structure of a selected molecule. The method can include providing, by the one or more computing devices, the input data that describes the chemical structure of the selected molecule as input to the machine-learned sensory prediction model. The method can include receiving, by the one or more computing devices, prediction data descriptive of one or more second sensory properties of the selected molecule associated with a second sensory prediction task as an output of the machine-learned sensory prediction model. The method can include providing, by the one or more computing devices, the prediction data descriptive of the one or more second sensory properties of the selected molecule as an output.

Another example aspect of the present disclosure is directed to one or more non-transitory computer-readable media comprising a sensory embedding, the sensory embedding generated as output from a machine-learned embedding model, wherein the machine-learned embedding model is trained using a first sensory prediction task training dataset for a first sensory prediction task and a second sensory prediction task training dataset for a second sensory prediction task, wherein a number of data items of the first sensory prediction task training dataset is greater than a number of data items of the second sensory prediction task training dataset.

Another example aspect of the present disclosure is directed to a composition of matter having a molecular structure designed based at least in part on a sensory embedding to exhibit one or more desired sensory properties, the sensory embedding generated as output from a machine-learned embedding model in response to receipt of input data descriptive of the molecular structure, wherein the machine-learned embedding model is trained using a first sensory prediction task training dataset for a first sensory prediction task and the embedding is used for a second sensory prediction task.

Other aspects of the present disclosure are directed to various systems, apparatuses, non-transitory computer-readable media, user interfaces, and electronic devices.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

Detailed discussion of embodiments directed to one of ordinary skill in the art is set forth in the specification, which makes reference to the appended figures, in which:

FIG. 1A depicts a block diagram of an example computing system according to example embodiments of the present disclosure.

FIG. 1B depicts a block diagram of an example computing device according to example embodiments of the present disclosure.

FIG. 1C depicts a block diagram of an example computing device according to example embodiments of the present disclosure.

FIG. 2 depicts a block diagram of an example prediction model according to example embodiments of the present disclosure.

FIG. 3 depicts a block diagram of an example prediction model according to example embodiments of the present disclosure.

FIG. 4 depicts a flowchart diagram of an example method for predicting sensory properties for a prediction task having limited available training data according to example embodiments of the present disclosure.

FIG. 5 depicts a flowchart diagram of an example method for training a sensory prediction model predicting sensory properties for a prediction task having limited available training data according to example embodiments of the present disclosure.

FIG. 6 depicts example illustrations for visualizing structural contribution associated with predicted sensory properties (e.g., olfactory properties) according to example embodiments of the present disclosure.

FIG. 7 illustrates an example model schematic and data flow according to example embodiments of the present disclosure.

FIG. 8A illustrates the global structure of an example learned embedding space according to example embodiments of the present disclosure.

FIG. 8B illustrates the global structure of an example learned embedding space according to example embodiments of the present disclosure.

Reference numerals that are repeated across plural figures are intended to identify the same features in various implementations.

### DETAILED DESCRIPTION

Example aspects of the present disclosure are directed to systems and methods that include or otherwise leverage machine-learned models (e.g., graph neural networks) in conjunction with molecular chemical structure data to predict one or more sensory and/or perceptual (e.g., olfactory, gustatory, tactile, etc.) properties of a molecule. In particular, the systems and methods of the present disclosure can include models (e.g., embedding models) that are trained for a first sensory prediction task based on the chemical structure of the molecule. At least a portion of the model can then be used for a second sensory prediction task that is different from the first sensory prediction task. In some implementations, the second sensory prediction task may be significantly different from the first sensory prediction task, such as, for example, a sensory prediction task related to a different species, different sense, different application, etc. from the first sensory prediction task. For instance, the first sensory task can be a sensory task for which a larger amount of training data is available than for the second sensory task. As one example, the first sensory task may be a sensory task related to human perception (e.g., human olfactory senses) and the second sensory task may be a sensory task related to a nonhuman species' perception. For example, a greater amount of training data may be available for human senses than for other species' senses. Unexpectedly, however, many sensory prediction tasks exhibit great transferability to seemingly unrelated or divergent sensory prediction tasks.

More particularly, the relationship between a molecule's structure and its olfactory and/or other sensory (e.g., gustatory) perceptual properties (e.g., the scent of a molecule as observed by a human) is complex, and, to date, generally little is known about such relationships. While some properties of molecules (e.g., materials properties, pharmaceutical properties, etc.) can have directly predictable attributes, olfactory, gustatory, and/or other perceptual or sensory properties can be a combination of molecular structure, receptor structure, concentration, bases, and/or other factors, which can greatly complicate modeling and simulation.

This challenge can be compounded by a lack of available data for some sensory characteristics. For instance, human and/or animal response data may be required to design molecules for these applications, and this data can be heavily limited in some domains. For instance, some particular sensory properties may have limited available data for humans and/or other species. As another example, comparatively large datasets may be available in one domain, such as human scent perception, but little might be available for another, such as mosquito repellents.

One existing solution to overcome this problem is use of a non-learned general purpose descriptor (e.g. SMILES string, Morgan fingerprint, Dragon descriptor, etc.). These descriptors are generally intended to “featurize” a molecule, rather than convey complicated structural interrelations. For instance, some existing approaches featurize or represent the molecule with general purpose heuristic features, such as Morgan fingerprints or Dragon descriptors. However, the general purpose featurization strategies often do not highlight the important information related to specific tasks, such as predicting the olfactory or other sensory properties of molecules in a given species. For instance, Morgan fingerprints are generally designed for “lookup” of similar molecules. Morgan fingerprints generally do not include spatial arrangement of a molecule. While this information can nonetheless be useful, it may be insufficient alone, in some design cases, such as olfactory cases which may benefit from spatial understanding. Despite this, a scratch-trained model with a low amount of available training data is unlikely to beat a Morgan fingerprint model.

Another existing approach is physics-based modeling of sensory properties. For instance, physics-based modeling can include computational modeling of sensory (e.g., olfactory) receptors or sensory-related (e.g., olfactory-related) proteins. For instance, given a computational model of the olfactory receptor target, it is possible to run high throughput docking screens to find molecular candidates for desired tasks. However, this can be complicated for certain tasks, as it can be computationally expensive to model all possible interactions for all candidates. Furthermore, physics-based modeling of sensory performance can require explicit knowledge about the task at hand, such as the physical structure of a receptor, its binding pocket, and the positioning of a chemical ligand in that pocket, which may not be readily available. Furthermore, while some properties (e.g., pharmaceutical properties, material properties) of a molecule may be easily learned, some sensory/perception properties in particular, such as sensory properties (e.g., olfactory properties), can be challenging to make predictions for. This can be further complicated by the fact that a base, such as ethanol, plastic, shampoo, soap, fabric, etc., for certain scented chemicals can affect perceived smell of the chemical. For instance, the same chemical may be perceived differently in an ethanol base compared to, for example, a soap base. Thus, even for chemicals that have a large amount of available training data in one base, there may be a limited amount of data in another base.

For example, in the domain of insect repellents, some potential repellants may act either as antagonists or as secondary inhibitors, and it would be computationally expensive to model each possible interaction. In addition, the physical structure of only many sensory receptors may be unavailable, which lends that traditional docking simulation may be impossible. For instance, from the insect repellent screening perspective, existing methods used to predict chemical properties involve simulating the docking of a specific molecule in a receptor pocket via detailed molecular dynamics simulation or binding mode prediction. However, these methods require expensive or difficult-to-acquire prior data in order to function in a new domain, such as the crystal structure of a specific receptor to be bound. Since perception (e.g., scent, taste) is the result of the collaborative activation of many hundreds of receptor types, and the crystal structure of very few receptors involved in chemical perception are known, this approach is often not possible or overly complicated.

Example aspects of the present disclosure can provide solutions for these and other challenges. According to an aspect of the present disclosure, a machine-learned sensory prediction model may be trained on a first sensory prediction task and used to output predictions associated with a second sensory prediction task. As one example, the first sensory prediction task may be a broader sensory prediction task than the second sensory prediction task. For example, the model may be trained on a broad task and transferred to a narrow task. As one example, the first task may be a broad property task and the second task may be a specific property task (e.g., olfactory). Additionally and/or alternatively, the first sensory prediction task may be a task for which a larger amount of training data is available than for the second sensory prediction task. Additionally and/or alternatively, the first sensory prediction task may be associated with a first species and the second sensory prediction task may be associated with a second species. As one example, the first sensory prediction task may be a human olfactory task. Additionally and/or alternatively, the second sensory prediction task may be a pest control task, such as a mosquito repellent task.

Additionally and/or alternatively, in some implementations, a machine-learned graph neural network can be trained and used to process a graph that graphically describes the chemical structure of a molecule to predict sensory properties (e.g., olfactory properties) of the molecule. In particular, the graph neural network can operate directly upon the graph representation of the chemical structure of the molecule (e.g., perform convolutions within the graph space) to predict the olfactory or other sensory properties of the molecule. As one example, the graph can include nodes that correspond to atoms and edges that correspond to chemical bonds between the atoms. Thus, the systems and methods of the present disclosure can provide prediction data that predicts sensory characteristics of molecules (e.g., for a second prediction task) through the use of machine-learned models.

As one example, a sensory embedding model can be trained to produce a sensory embedding for the first sensory prediction task. The sensory embedding can be learned from the first sensory prediction task, such as from a larger available dataset, such that the sensory embedding is specific to the first prediction task (e.g., a broader task). Despite being trained with regard to the first prediction task, however, it is recognized according to example aspects of the present disclosure that this sensory embedding can capture useful information for other (e.g., narrower) sensory prediction tasks. Furthermore, this sensory embedding can be transferred, fine-tuned or otherwise modified to produce accurate predictions in another domain for the second sensory prediction task that has less available data then the first sensory prediction task, such as a task where machine learning or accurate prediction would otherwise be difficult and/or impossible.

As one example, a sensory embedding model can be trained in tandem with a first prediction task model. The sensory embedding model and the first prediction task model can be trained using (e.g., labeled) first prediction task training data for the first prediction task. For instance, the sensory embedding model can be trained to produce sensory embeddings with respect to the first prediction task. These sensory embeddings can capture information that is useful in the second prediction task. After training the sensory embedding model with the first prediction task model on first prediction task training data, the sensory embedding model can be used with a second prediction task model to output predictions associated with the second prediction task. In some cases, the sensory embedding model can further be refined, fine-tuned, or otherwise continually trained on second prediction task training data associated with the second prediction task. In some implementations, the model may be trained at a lower training rate with the second prediction task than for the first prediction task, to prevent intuitively un-learning the information learned from the first prediction task. In some implementations, an amount of second prediction task training data may be less than an amount of first prediction task training data, such as if there is less available data for the second prediction task than for the first prediction task.

The machine-learned models can be trained, for example, using training data that includes descriptions of molecules (e.g., structural descriptions of molecules, graph-based descriptions of chemical structures of molecules, etc.) for a first sensory prediction task, such as molecules that have been labeled (e.g., manually by an expert) with descriptions of sensory properties (e.g., olfactory properties) (e.g., textual descriptions of odor categories such as “sweet,” “piney,” “pear,” “rotten,” etc.) that have been assessed for the molecules. For instance, these descriptions of olfactory molecules may relate to, for example, human perception. These models can then be used for a second sensory prediction task that is different from the first sensory prediction task. For instance, the second sensory prediction task may relate to non-human perception. For instance, in some implementations, the model is transferred across different species' perceptual properties of molecules.

In this way, a model that is trained on a large dataset can be transferred to a task having a smaller dataset while still achieving high predictive performance. In particular, it is observed that the sensory embeddings can provide a significant boost to prediction quality when transfer learning across species for sensory (e.g., olfactory) prediction tasks. Beyond even in-domain transfer learning, these sensory embeddings can provide improved performance for even more disparate qualities, such as cross-species perception. This is especially unexpected in the chemical domain. For instance, the sensory embeddings may be taken directly as input at a second prediction task model. The sensory embedding model may then be fine-tuned and trained on the second sensory prediction task. Unexpectedly, the second sensory prediction task and the first sensory prediction task need not be overly similar. For instance, prediction tasks having sufficient distinction (e.g., cross-species, cross-domain, etc.) may nonetheless find benefit according to example aspects of the present disclosure.

Thus, some example aspects of the present disclosure are directed to propose the use of neural networks, such as graph neural networks, for olfactory, gustatory, and/or other sensory modeling across distinct domains, such as quantitative structure-odor relationship (QSOR) modeling. Graph neural networks can represent spatial information, which can be important for olfactory and/or other sensory modeling. Example implementations of the systems and methods described herein significantly outperform prior methods on a novel data set labeled by olfactory experts. Furthermore, the learned sensory embeddings from graph neural networks capture a meaningful odor space representation of the underlying relationship between structure and odor. These learned sensory embeddings can unexpectedly be applied to domains other than the domain for which the model used to generate the sensory embedding is learned. For example, a model trained on human sensory perception data may unexpectedly achieve desirable results outside of the human sensory perception domain, such as other species' perception and/or other domains. For instance, the use of graph neural networks can provide spatial understanding to the model that is beneficial for sensory modeling applications.

More particularly, according to one aspect of the present disclosure, machine-learned models, such as graph neural network models, can be trained to provide predictions of perceptual properties (e.g., sensory properties (e.g., olfactory properties), gustatory properties, tactile properties, etc.) of a molecule based on an input representing qualities of the molecule, such as, for example, a graph of the chemical structure of the molecule. For instance, a machine-learned model may be provided with an input that is descriptive of the molecule, such as a graph structure of a molecule's chemical structure, for example, based on a standardized description of a molecule's chemical structure and/or qualities (e.g., a Morgan fingerprint, simplified molecular-input line-entry system (SMILES) string, etc.). The machine-learned model may provide output comprising a description of predicted perceptual properties of the molecule, such as, for example, a list of olfactory perceptual properties descriptive of what the molecule would smell like and/or performance at other olfactory or other sensory tasks (e.g., repellent). As another example, the model may be configured to produce a sensory embedding. The sensory embedding may then be used as input to a second prediction task model that is configured to provide the final output for the second sensory prediction task.

For instance, a SMILES string can be provided, such as the SMILES string “O=C(OCCC(C)C)C” for the chemical structure of isoamyl acetate, and the machine-learned model can provide as output a description of what that molecule would smell like to a human, for example, a description of the molecule's odor properties such as “fruit, banana, apple”. In particular, in some embodiments, in response to receipt of a SMILES string or other description of chemical structure, the systems and methods of the present disclosure can convert the string to a graph structure that graphically describes the two-dimensional structure of a molecule and can provide the graph structure to a machine-learned model (e.g., a trained graph convolutional neural network and/or other type of machine-learned model) that can predict, from either the graph structure or features derived from the graph structure, sensory properties (e.g., olfactory properties) of the molecule. Additionally or alternatively to the two-dimensional graph, systems and methods could provide for creating a three-dimensional graph representation of the molecule, for example using quantum chemical calculations, for input to a machine-learned model.

In some examples, the prediction for the first prediction task and/or the second prediction task can indicate whether or not the molecule has a particular desired sensory quality (e.g., a target scent perception, etc.). In some embodiments, the prediction data can include one or more types of information associated with a predicted sensory property (e.g., olfactory property) of a molecule. For instance, prediction data for a molecule can provide for classifying the molecule into one sensory property (e.g., olfactory property) class and/or into multiple sensory property (e.g., olfactory property) classes. In some instances, the classes can include human-provided (e.g., experts) textual labels (e.g., sour, cherry, piney, etc.). In some instances, the classes can include non-textual representations of scent/odor, such as a location on a scent continuum or the like. In some instances, prediction data for molecules can include intensity values that describe the intensity of the predicted scent/odor. In some instances, prediction data can include confidence values associated with the predicted olfactory perceptual property. As another example, in some implementations, the prediction data may be descriptive of how well the molecule will perform at a particular task (e.g., a pest control task).

In addition or alternatively to specific classifications for a molecule, prediction data can include a numerical sensory embedding that allows for similarity search, clustering, or other comparisons between two or more molecules based on a measure of distance between two or more sensory embeddings. For example, in some implementations, the machine-learned model can be trained to output sensory embeddings that can be used to measure similarity by training the machine-learned model using a triplet training scheme where the model is trained to output sensory embeddings that are closer in the sensory embedding space for a pair of similar chemical structures (e.g., an anchor example and a positive example) and to output sensory embeddings that are more distant in the sensory embedding space for a pair of dissimilar chemical structures (e.g., the anchor and a negative example). According to example aspects of the present disclosure, these output sensory embeddings may be used even in dissimilar tasks such as cross-species tasks.

Thus, in some implementations, the systems and methods of the present disclosure may not necessitate the generation of feature vectors descriptive of the molecule for input to a machine-learned model. Rather, the machine-learned model can be provided directly with the input of a graph-value form of the original chemical structure, thus reducing the resources required to make sensory property (e.g., olfactory property) predictions. For example, by providing for the use of the graph structure of molecules as input to the machine-learned model, new molecule structures can be conceptualized and evaluated without requiring the experimental production of such molecule structures to determine perceptual properties, thereby greatly accelerating the ability to evaluate new molecular structure and saving significant resources.

According to another aspect of the present disclosure, training data comprising a plurality of known molecules can be obtained to provide for training one or more machine-learned models (e.g., a graph convolutional neural network, other type of machine-learned model) to provide predictions of sensory properties (e.g., olfactory properties) of molecules. For example, in some embodiments, the machine-learned models can be trained using one or more datasets of molecules, where the dataset includes the chemical structure and a textual description of the perceptual properties (e.g., descriptions of the smell of the molecule provided by human experts, etc.) for each molecule. As one example, the training data can be derived from publicly available data such as, for example, publicly available lists of chemical structures and their corresponding odors. In some embodiments, due to the fact that some perceptual properties are rare, steps can be taken to balance out common perceptual properties and rare perceptual properties when training the machine-learned model(s). According to example aspects of the present disclosure, the training data may be provided for a first sensory prediction task, where the training data is more widely available than for a second sensory prediction task that is an overall objective of the model. The model may then be retrained for the second sensory prediction task on a (limited) amount of training data for the second sensory prediction task and/or used as-is for the second sensory prediction task without further training.

According to another aspect of the present disclosure, in some embodiments, the systems and methods may provide for indications of how changes to a molecule structure could affect the predicted perceptual properties (e.g., for the second prediction task). For example, the systems and methods could provide indications of how changes to the molecule structure may affect the intensity of a particular perceptual property, how catastrophic a change in the molecule's structure would be to desired perceptual qualities, and/or the like. In some embodiments, the systems and methods may provide for adding and/or removing one or more atoms and/or groups of atoms from a molecule's structure to determine the effect of such addition/removal on one or more desired perceptual properties. For example, iterative and different changes to the chemical structure can be performed and then the result can be evaluated to understand how such change would affect the perceptual properties of the molecule. As yet another example, a gradient of the classification function of the machine-learned model can be evaluated (e.g., with respect to a particular label) at each node and/or edge of the input graph (e.g., via backpropagation through the machine-learned model) to generate a sensitivity map (e.g., that indicates how important each node and/or edge of the input graph was for output of such particular label). Further, in some implementations, a graph of interest can be obtained, similar graphs can be sampled by adding noise to the graph, and then the average of the resulting sensitivity maps for each sampled graph can be taken as the sensitivity map for the graph of interest. Similar techniques can be performed to determine perceptual differences between different molecular structures.

According to another aspect, the systems and methods of the present disclosure can provide for interpreting and/or visualizing which aspects of a molecule's structure most contributes to a predicted sensory quality (e.g., for the second prediction task). For example, in some embodiments, a heat map could be generated to overlay the molecule structure that provides indications of which portions of a molecule's structure are most important to the perceptual properties of the molecule and/or which portions of a molecule's structure are less important to the perceptual properties of the molecule. In some implementations, data indicative of how changes to a molecule structure would impact olfactory perception can be used to generate visualizations of how the structure contributes to a predicted olfactory quality. For example, as described above, iterative changes to the molecule's structure (e.g., a knock-down technique, etc.) and their corresponding outcomes can be used to evaluate which portions of the chemical structure are most contributory to the olfactory perception. As another example, as described above, a gradient technique can be used to generate a sensitivity map for the chemical structure, which can then be used to produce the visualization (e.g., in the form of a heat map).

According to another aspect of the present disclosure, in some embodiments, machine-learned model(s) may be trained to produce predictions of a molecule chemical structure that would provide one or more desired perceptual properties (e.g., generate a molecule chemical structure that would produce a particular scent quality, etc.). For example, in some implementations, an iterative search can be performed to identify proposed molecule(s) that are predicted to exhibit one or more desired perceptual properties (e.g., targeted scent quality, intensity, etc.). For instance, an iterative search can propose a number of candidate molecule chemical structures that can be evaluated by the machine-learned model(s). In one example, candidate molecule structures can be generated through an evolutionary or genetic process. As another example, candidate molecule structures can be generated by a reinforcement learning agent (e.g., recurrent neural network) that seeks to learn a policy that maximizes a reward that is a function of whether the generated candidate molecule structures exhibit the one or more desired perceptual properties. According to example aspects of the present disclosure, this perceptual property analysis can be related to a second sensory prediction task that is different from the first sensory prediction task.

Thus, in some implementations, a plurality of candidate molecule graph structures that describe the chemical structure of each candidate molecule can be generated (e.g., iteratively generated) for use as input to a machine-learned model. The graph structure for each candidate molecule can be input to the machine-learned model to be evaluated. The machine-learned model can produce prediction data for each candidate molecule that describes one or more perceptual properties of the candidate molecule. The candidate molecule prediction data can then be compared to the one or more desired perceptual properties to determine if the candidate molecule would exhibit desired perceptual properties (e.g., a viable molecule candidate, etc.). For example, the comparison can be performed to generate a reward (e.g., in a reinforcement learning scheme) or to determine whether to retain or discard the candidate molecule (e.g., in an evolutionary learning scheme). These results may further be used to train the model. Brute force search approaches may also be employed. In further implementations, which may or may not have the evolutionary or reinforcement learning structures described above, the search for candidate molecules that exhibit the one or more desired perceptual properties can be structured as a multi-parameter optimization problem with a constraint on the optimization defined for each desired property.

According to another aspect of the present disclosure, systems and methods may provide for predicting, identifying, and/or optimizing other properties associated with a molecule structure along with desired sensory properties (e.g., olfactory properties). For example, the machine-learned model(s) may predict or identify properties of molecule structures such as optical properties (e.g., clarity, reflectiveness, color, etc.), olfactory properties (e.g., scents such as scents reminiscent of scents of fruits, flowers, etc.), gustatory properties (e.g., tastes like “banana,” “sour,” “spicy,” etc.) shelf-stability, stability at particular pH levels, biodegradability, toxicity, industrial applicability, and/or the like for a second sensory prediction task that is different from a first sensory prediction task on which the model(s) were earlier trained.

According to another aspect of the present disclosure, the machine-learned models described herein can be used in active learning techniques to narrow a wide field of candidates to a smaller set of molecules that are then manually evaluated. According to other aspects of the present disclosure, systems and methods can allow for synthesis of molecules with particular properties in an iterative design-test-refine process. For example, based on prediction data from the machine-learned models, molecules can be proposed for development. The molecules can then be synthesized, and then can be subjected to specialized testing. Feedback from the testing can then be provided back to the design phase to refine the molecules to better achieve desired properties, etc. For example, results from the testing can be used as training data to re-train the machine-learned model. After re-training, predictions form the model can then again be used to identify certain molecules for testing. Thus, an iterative pipeline can be evaluated where a model is used to select candidates and then testing results for the candidates can be used to re-train the model, and so on.

For instance, in one example implementation of the present disclosure, a model is trained using a large amount of human perceptual data, which may be readily available as training data. The model is then transferred to an at least somewhat related chemical problem, such as predicting whether a molecule will be a good mosquito repellent, discovering a new flavor molecule, etc. The model (e.g., a neural network) can also be packaged into a standalone molecule embedding tool for generating representations that focus on olfactory related problems. These representations can be used to search for odors that smell similarly or trigger similar behavior in animals. The embedding space described herein can additionally be useful as a codec for designing electronic scent perception systems (e.g., “electronic noses”).

The systems and methods of the present disclosure provide a number of technical effects and benefits. As one example, the systems and methods described herein can allow for reducing the time and resources required to determine whether a molecule would provide desired perceptual qualities. For instance, the systems and methods described herein allow for using graph structures descriptive of the chemical structure of a molecule rather than necessitating the generation of feature vectors describing a molecule to provide for model input. Thus, the systems and methods provide technical improvements in the resources required to obtain and analyze model inputs and produce model prediction outputs. Furthermore, the use of machine-learned models to predict sensory properties (e.g., olfactory properties) represents the integration of machine learning into a practical application (e.g., predicting sensory properties (e.g., olfactory properties)). That is, the machine-learned models are adapted to the specific technical implementation of predicting sensory properties (e.g., olfactory properties). The machine-learned models according to example aspects of the present disclosure can further significantly outperform existing systems, including, unexpectedly, in domains that do not include great amounts of training data.

The use of sensory property prediction and modeling can find application among a variety of fields or tasks. As one example, designing molecules for certain sensory properties can be a particularly demanding challenge in designing fragranced goods, such as lotions, shampoos, perfumes, etc. For instance, in some implementations, the first sensory prediction task can be a human olfactory task related to predicting olfactory perceptual properties for humans, such as labels that describe what a molecule smells like. For example, in some implementations, the first sensory prediction task and/or the second sensory prediction task can be a human olfactory task. The sensory properties can be human olfactory perception properties, such as what a molecule smells like. The second sensory prediction task may be a human olfactory task in a different context from the first sensory prediction task, such as what a molecule will smell like in a different chemical base. For example, the first sensory prediction task may relate to predicting sensory properties in a first base (e.g., ethanol) for which training data is more readily available while the second sensory prediction task may relate to predicting sensory properties (e.g., for a same sense) in a second base (e.g., soap, lotion, etc.) which may have less available data.

As another example, certain sensory properties can be desirable for animal attractant and/or repellent tasks. For instance, the first sensory prediction task can be a human sensory task, such as human olfactory task, a human gustatory task, etc., based on chemical structure of a molecule. The first sensory property can be human perception properties, such as human olfactory perceptual properties and/or human gustatory perceptual properties. The second sensory prediction task can be a nonhuman sensory task, such as a related sensory task for another species. The second sensory prediction task can additionally and/or alternatively be or include performance of the molecule as an attractant and/or repellent for a certain species. For instance, the properties may indicate performance of the molecule at attracting a desired species (e.g., for incorporation into animal food, etc.), or repelling undesired species (e.g., an insect repellent).

For example, this can include pest control applications, such as mosquito repellent, insecticides, etc. For example, mosquito repellent may serve to repel mosquitos and prevent bites contributing to transmission of viruses and diseases. For instance, services or technologies that relate to human and/or animal olfactory systems could potentially find use for systems and methods according to example aspects in various implementations. Example implementations can include, for example, approaches for finding suitable odors for insect repellent or other pest control, such as repellent for mosquitos, pests that affect crop health, livestock health, personal health, building/infrastructure health, and/or other suitable pests. For instance, systems and methods described herein may be useful for designing a repellent, insecticide, attractant, etc. for a targeted species of insect or other animal, even animals for which little to no sensory perception data is available. As one example, the first sensory prediction task can be a sensory prediction task related to a human sense, such as a human olfactory task of predicting human olfactory perception labels based on molecular structure data. The second sensory prediction task may include predicting performance of molecules at repelling another species, such as mosquitos.

As another example, systems and methods according to example aspects of the present disclosure may find application in toxicology and/or other safety studies. For example, the first sensory prediction task and/or the second sensory prediction task may be toxicology prediction tasks. The sensory properties may relate to toxicity of chemicals based on chemical structures. As another example, systems and methods according to example aspects of the present disclosure can be beneficial in transferring to related olfactory tasks, such as discovering a molecule that will smell similar to an existing molecule, but with different physical properties such as color.

In some embodiments, the systems and methods described herein can be implemented by one or more computing devices. the computing device(a) can include one or more processors and one or more non-transitory computer-readable media that store instructions that, when executed by the one or more processors, cause the computing device to perform operations. The operations can include steps of various methods described herein.

Example aspects of the present disclosure are discussed with reference to molecular structures. It should be understood by one of ordinary skill in the art that example aspects of the present disclosure can be extended to molecular mixtures including a plurality of unique molecular structures. For instance, in some implementations, a mixture can be represented as variable size sets of modules with corresponding ratios by weight and volume. This representation may also include order of composition, process steps, etc. In some implementations, each molecule in the mixture can be a unique graph. Additionally and/or alternatively, a graph representing the mixture can include nodes corresponding to individual molecules and/or edges defining interactions between the molecules. The model can be trained for a prediction task, such as to learn interactions between a limited library of available molecules.

With reference now to the Figures, example embodiments of the present disclosure will be discussed in further detail.

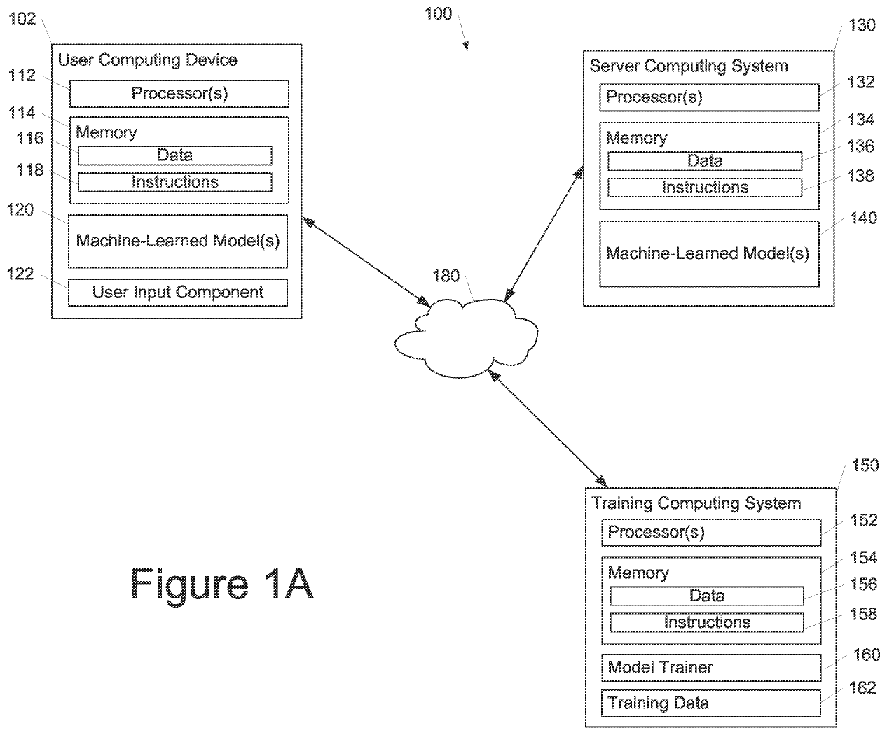

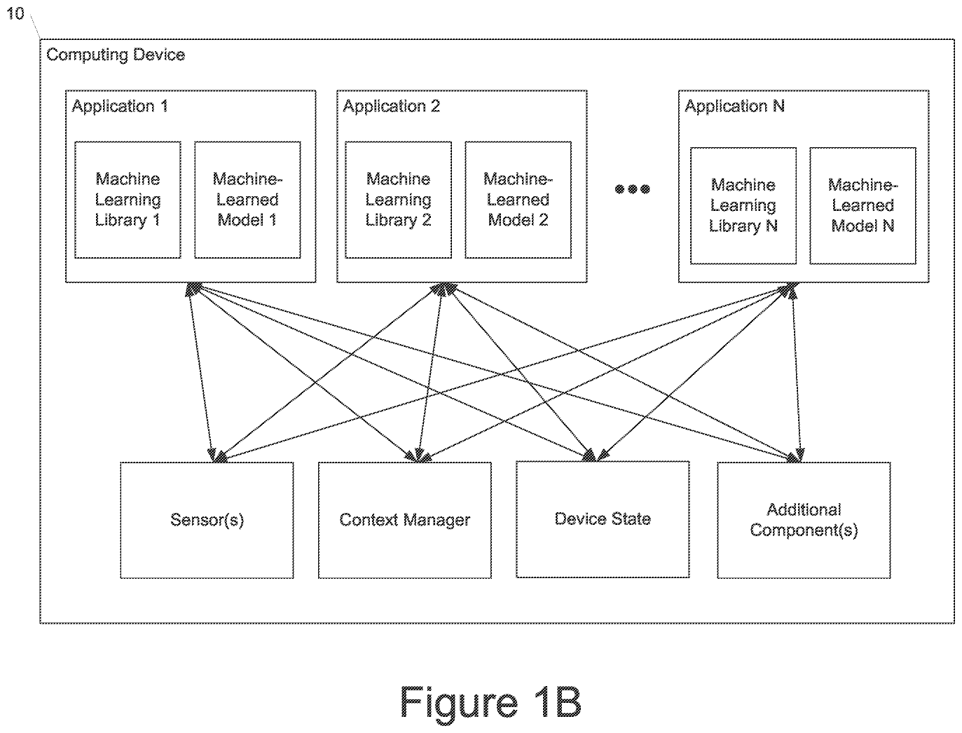

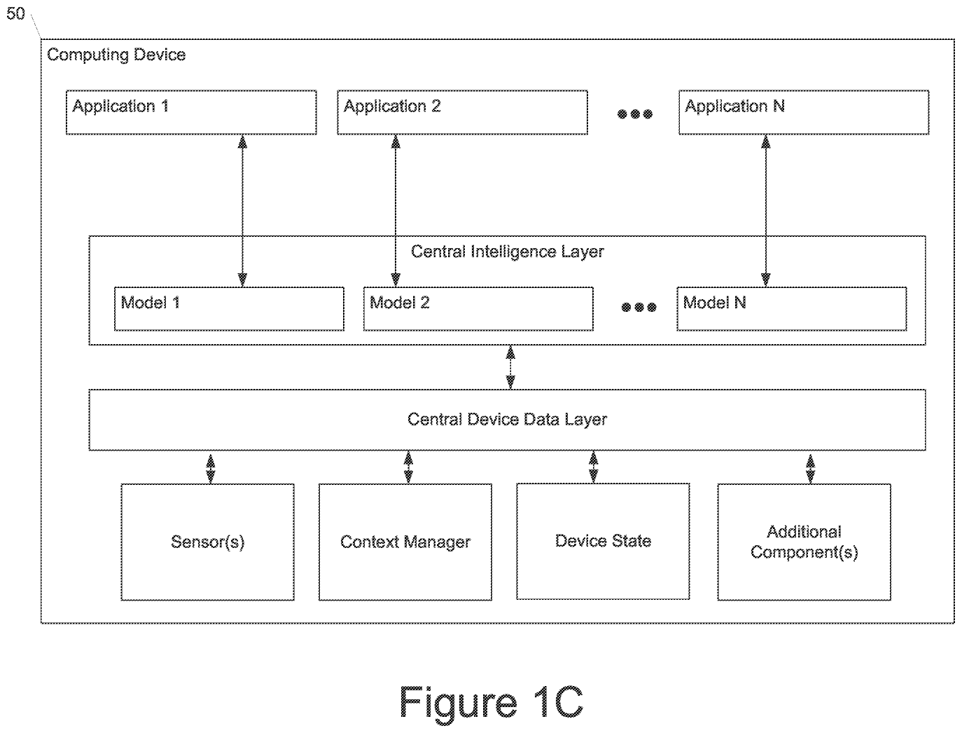

FIG. 1A depicts a block diagram of an example computing system 100 that can facilitate predictions of sensory properties, such as olfactory sensory properties, of molecules according to example embodiments of the present disclosure. The system 100 is provided as one example only. Other computing systems that include different components can be used in addition or alternatively to the system 100. The system 100 includes a user computing device 102, a server computing system 130, and a training computing system 150 that are communicatively coupled over a network 180.

The user computing device 102 can be any type of computing device, such as, for example, a personal computing device (e.g., laptop or desktop), a mobile computing device (e.g., smartphone or tablet), a gaming console or controller, a wearable computing device, an embedded computing device, or any other type of computing device.

The user computing device 102 includes one or more processors 112 and a memory 114. The one or more processors 112 can be any suitable processing device (e.g., a processor core, a microprocessor, an ASIC, a FPGA, a controller, a microcontroller, etc.) and can be one processor or a plurality of processors that are operatively connected. The memory 114 can include one or more non-transitory computer-readable storage mediums, such as RAM, ROM, EEPROM, EPROM, flash memory devices, magnetic disks, etc., and combinations thereof. The memory 114 can store data 116 and instructions 118 which are executed by the processor 112 to cause the user computing device 102 to perform operations.

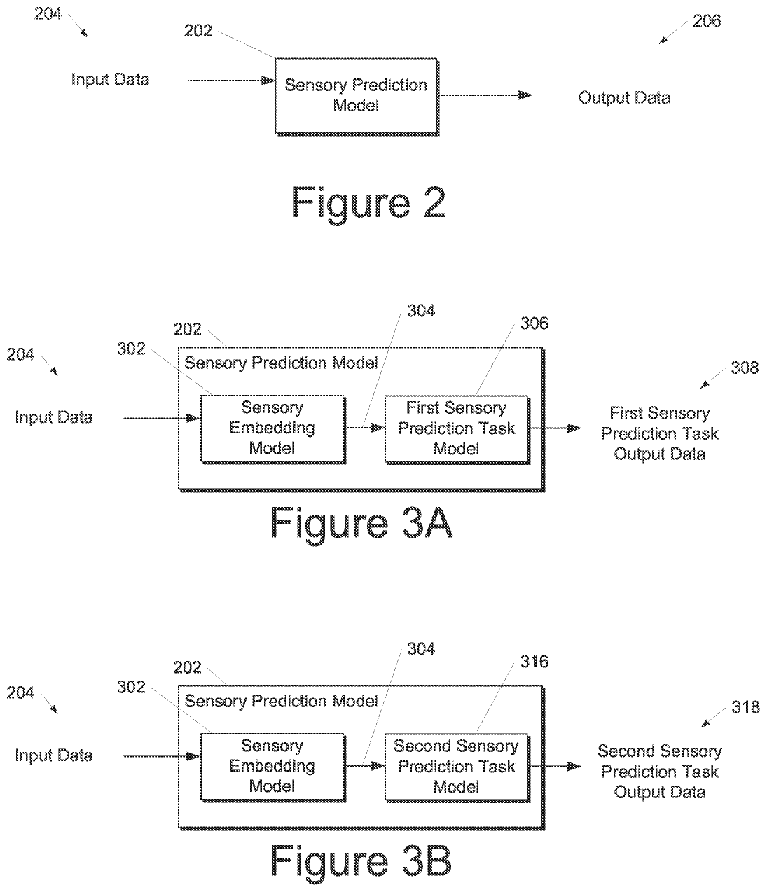

In some implementations, the user computing device 102 can store or include one or more machine-learned models 120, such as a sensory property (e.g., olfactory property) prediction machine-learned model as discussed herein. For example, the machine-learned models 120 can be or can otherwise include various machine-learned models such as neural networks (e.g., deep neural networks) or other types of machine-learned models, including non-linear models and/or linear models. Neural networks can include feed-forward neural networks, recurrent neural networks (e.g., long short-term memory recurrent neural networks), convolutional neural networks or other forms of neural networks. Example machine-learned models 120 are discussed with reference to FIGS. 2 and 3.

In some implementations, the one or more machine-learned models 120 can be received from the server computing system 130 over network 180, stored in the user computing device memory 114, and then used or otherwise implemented by the one or more processors 112. In some implementations, the user computing device 102 can implement multiple parallel instances of a single machine-learned model 120.

Additionally or alternatively, one or more machine-learned models 140 can be included in or otherwise stored and implemented by the server computing system 130 that communicates with the user computing device 102 according to a client-server relationship. For example, the machine-learned models 140 can be implemented by the server computing system 140 as a portion of a web service. Thus, one or more models 120 can be stored and implemented at the user computing device 102 and/or one or more models 140 can be stored and implemented at the server computing system 130.

The user computing device 102 can also include one or more user input component 122 that receives user input. For example, the user input component 122 can be a touch-sensitive component (e.g., a touch-sensitive display screen or a touch pad) that is sensitive to the touch of a user input object (e.g., a finger or a stylus). The touch-sensitive component can serve to implement a virtual keyboard. Other example user input components include a microphone, a traditional keyboard, a camera, or other means by which a user can provide user input.

The server computing system 130 includes one or more processors 132 and a memory 134. The one or more processors 132 can be any suitable processing device (e.g., a processor core, a microprocessor, an ASIC, a FPGA, a controller, a microcontroller, etc.) and can be one processor or a plurality of processors that are operatively connected. The memory 134 can include one or more non-transitory computer-readable storage mediums, such as RAM, ROM, EEPROM, EPROM, flash memory devices, magnetic disks, etc., and combinations thereof. The memory 134 can store data 136 and instructions 138 which are executed by the processor 132 to cause the server computing system 130 to perform operations.

In some implementations, the server computing system 130 includes or is otherwise implemented by one or more server computing devices. In instances in which the server computing system 130 includes plural server computing devices, such server computing devices can operate according to sequential computing architectures, parallel computing architectures, or some combination thereof.

As described above, the server computing system 130 can store or otherwise include one or more machine-learned models 140. For example, the models 140 can be or can otherwise include various machine-learned models, such as sensory property (e.g., olfactory property) prediction machine-learned models. Example machine-learned models include neural networks or other multi-layer non-linear models. Example neural networks include feed forward neural networks, deep neural networks, recurrent neural networks, and convolutional neural networks. Example models 140 are discussed with reference to FIGS. 2 through 4.

The user computing device 102 and/or the server computing system 130 can train the models 120 and/or 140 via interaction with the training computing system 150 that is communicatively coupled over the network 180. The training computing system 150 can be separate from the server computing system 130 or can be a portion of the server computing system 130.

The training computing system 150 includes one or more processors 152 and a memory 154. The one or more processors 152 can be any suitable processing device (e.g., a processor core, a microprocessor, an ASIC, a FPGA, a controller, a microcontroller, etc.) and can be one processor or a plurality of processors that are operatively connected. The memory 154 can include one or more non-transitory computer-readable storage mediums, such as RAM, ROM, EEPROM, EPROM, flash memory devices, magnetic disks, etc., and combinations thereof. The memory 154 can store data 156 and instructions 158 which are executed by the processor 152 to cause the training computing system 150 to perform operations. In some implementations, the training computing system 150 includes or is otherwise implemented by one or more server computing devices.

The training computing system 150 can include a model trainer 160 that trains the machine-learned models 120 and/or 140 stored at the user computing device 102 and/or the server computing system 130 using various training or learning techniques, such as, for example, backwards propagation of errors. In some implementations, performing backwards propagation of errors can include performing truncated backpropagation through time. The model trainer 160 can perform a number of generalization techniques (e.g., weight decays, dropouts, etc.) to improve the generalization capability of the models being trained.

In particular, the model trainer 160 can train the machine-learned models 120 and/or 140 based on a set of training data 162. The training data 162 can include, for example, descriptions of molecules (e.g., graphical descriptions of chemical structures of molecules) that have been labeled (e.g., manually by an expert) with descriptions of sensory properties (e.g., olfactory properties) (e.g., textual descriptions of odor categories such as “sweet,” “piney,” “pear,” “rotten,” etc.) that have been assessed for the molecules, and/or the like. The model trainer 160 may train the models 120 and/or 140 using training data for the first prediction task and/or the second prediction task.

The model trainer 160 includes computer logic utilized to provide desired functionality. The model trainer 160 can be implemented in hardware, firmware, and/or software controlling a general purpose processor. For example, in some implementations, the model trainer 160 includes program files stored on a storage device, loaded into a memory, and executed by one or more processors. In other implementations, the model trainer 160 includes one or more sets of computer-executable instructions that are stored in a tangible computer-readable storage medium such as RAM hard disk or optical or magnetic media.

The network 180 can be any type of communications network, such as a local area network (e.g., intranet), wide area network (e.g., Internet), or some combination thereof and can include any number of wired or wireless links. In general, communication over the network 180 can be carried via any type of wired and/or wireless connection, using a wide variety of communication protocols (e.g., TCP/IP, HTTP, SMTP, FTP), encodings or formats (e.g., HTML, XML), and/or protection schemes (e.g., VPN, secure HTTP, SSL).

FIG. 1A illustrates one example computing system that can be used to implement the present disclosure. Other computing systems can be used as well. For example, in some implementations, the user computing device 102 can include the model trainer 160 and the training dataset 162. In such implementations, the models 120 can be both trained and used locally at the user computing device 102. Any components illustrated as being included in one of device 102, system 130, and/or system 150 can instead be included at one or both of the others of device 102, system 130, and/or system 150.

FIG. 1B depicts a block diagram of an example computing device 10 according to example embodiments of the present disclosure. The computing device 10 can be a user computing device or a server computing device.

The computing device 10 includes a number of applications (e.g., applications 1 through N). Each application contains its own machine learning library and machine-learned model(s). For example, each application can include a machine-learned model. Example applications include a text messaging application, an email application, a dictation application, a virtual keyboard application, a browser application, etc.

As illustrated in FIG. 1B, each application can communicate with a number of other components of the computing device, such as, for example, one or more sensors, a context manager, a device state component, and/or additional components. In some implementations, each application can communicate with each device component using an API (e.g., a public API). In some implementations, the API used by each application is specific to that application.

FIG. 1C depicts a block diagram of an example computing device 50 according to example embodiments of the present disclosure. The computing device 50 can be a user computing device or a server computing device.

The computing device 50 includes a number of applications (e.g., applications 1 through N). Each application is in communication with a central intelligence layer. Example applications include a text messaging application, an email application, a dictation application, a virtual keyboard application, a browser application, etc. In some implementations, each application can communicate with the central intelligence layer (and model(s) stored therein) using an API (e.g., a common API across all applications).

The central intelligence layer includes a number of machine-learned models. For example, as illustrated in FIG. 1C, a respective machine-learned model (e.g., a model) can be provided for each application and managed by the central intelligence layer. In other implementations, two or more applications can share a single machine-learned model. For example, in some implementations, the central intelligence layer can provide a single model (e.g., a single model) for all of the applications. In some implementations, the central intelligence layer is included within or otherwise implemented by an operating system of the computing device 50.

The central intelligence layer can communicate with a central device data layer. The central device data layer can be a centralized repository of data for the computing device 50. As illustrated in FIG. 1C, the central device data layer can communicate with a number of other components of the computing device, such as, for example, one or more sensors, a context manager, a device state component, and/or additional components. In some implementations, the central device data layer can communicate with each device component using an API (e.g., a private API).

FIG. 2 depicts a block diagram of an example prediction model 202 according to example embodiments of the present disclosure. In some implementations, the prediction model 202 is trained to receive a set of input data 204 (e.g., molecule chemical structure graph data, etc.) and, as a result of receipt of the input data 204, provide output data 206, for example, sensory property (e.g., olfactory property) prediction data for the molecule.

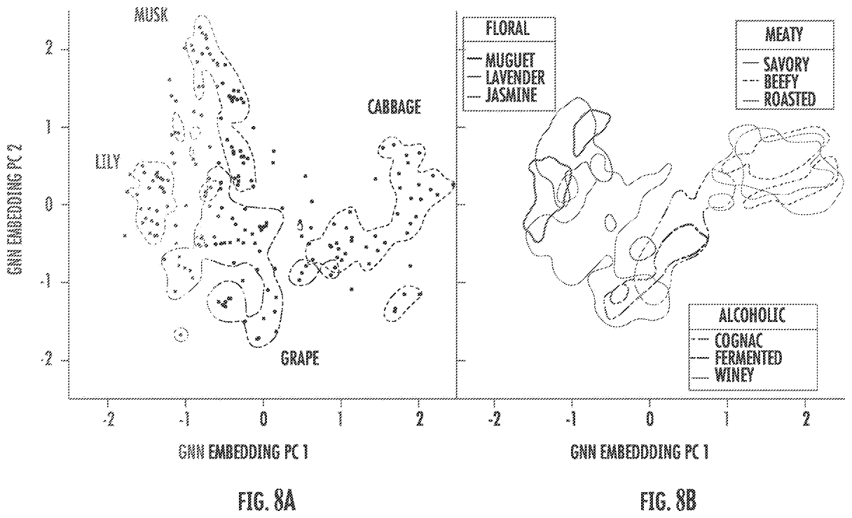

FIG. 3A depicts a block diagram of an example machine-learned model 202 according to example embodiments of the present disclosure. The machine-learned model 202 is similar to prediction model 202 of FIG. 2 except that machine-learned model 202 of FIG. 3A is one example model that includes a sensory embedding model 302. The sensory embedding model 302 can be configured to produce sensory embeddings 304 in response to receipt of input data 204. Example sensory embeddings 304 are discussed with reference to FIG. 8. The sensory embedding model 304 can be any suitable machine-learned model, such as a model including one or more neural networks, such as graph neural networks. As illustrated in FIG. 3A, the sensory embeddings 304 can be used as input to a first prediction task model 306 to produce first sensory prediction task output data 308. For instance, the sensory embeddings 304 can capture information that is useful for a first prediction task, which is achieved by use of first prediction task model 306. The first prediction task model 306 can be any suitable machine-learned model, such as, for example, a machine-learned model including one or more neural networks (e.g., graph neural networks). According to example aspects of the present disclosure, the sensory embedding model 302 may be trained by a first sensory prediction task training dataset for a first sensory prediction task while coupled to the first sensory prediction task model 306. Thus, the sensory embedding model 302 can be trained to generate sensory embeddings 304 for the first sensory prediction task.

FIG. 3B depicts a block diagram of an example machine-learned model 202 according to example embodiments of the present disclosure. The machine-learned model 202 of FIG. 3B is similar to the machine-learned model 202 of FIGS. 2 and 3A but includes a second prediction task model 316 configured to produce second sensory prediction task output data 318. For instance, according to example aspects of the present disclosure, once the sensory embedding model 302 is trained for a first sensory prediction task with first sensory prediction task model 306, the sensory embeddings 304 can be used as input to a second sensory prediction task model 316 for a second sensory prediction task. The sensory embedding model 302 may be trained for the second sensory prediction task based on a (e.g., limited) second sensory prediction task training dataset. The second sensory prediction task may represent an intended output of the sensory prediction model 202, and/or the first sensory prediction task may be a sensory task that is related to but distinct from the second sensory prediction task, such as a nearest task having a large amount of available training data.

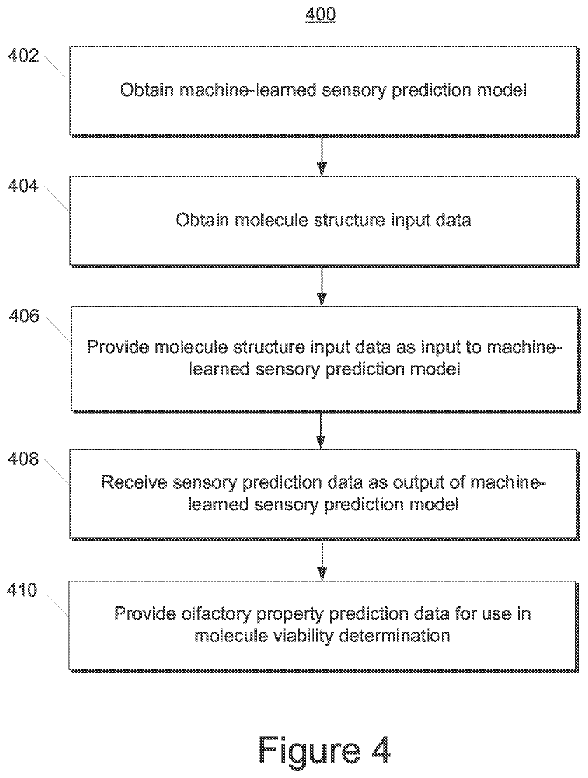

FIG. 4 depicts a flowchart diagram of an example method 400 for predicting sensory properties for a prediction task having limited available training data according to example embodiments of the present disclosure. Although FIG. 4 depicts steps performed in a particular order for purposes of illustration and discussion, the methods of the present disclosure are not limited to the particularly illustrated order or arrangement. The various steps of the method 400 can be omitted, rearranged, combined, and/or adapted in various ways without deviating from the scope of the present disclosure. Method 400 can be implemented by one or more computing devices, such as one or more of the computing devices depicted in FIGS. 1A-IC.

At 402, method 400 can include obtaining, by one or more computing devices, a machine-learned sensory prediction model (e.g., a graph neural network) trained to predict sensory properties (e.g., olfactory properties) of molecules based at least in part on chemical structure data associated with the molecules. In particular, a machine-learned prediction model (e.g., graph neural network, etc.) can be trained and used to process input data (e.g., a graph) that (e.g., graphically) describes the chemical structure of a molecule to predict sensory properties (e.g., olfactory properties) of the molecule. For example, a trained graph neural network can operate directly upon the graph representation of the chemical structure of the molecule (e.g., perform convolutions within the graph space) to predict the sensory properties (e.g., olfactory properties) of the molecule.

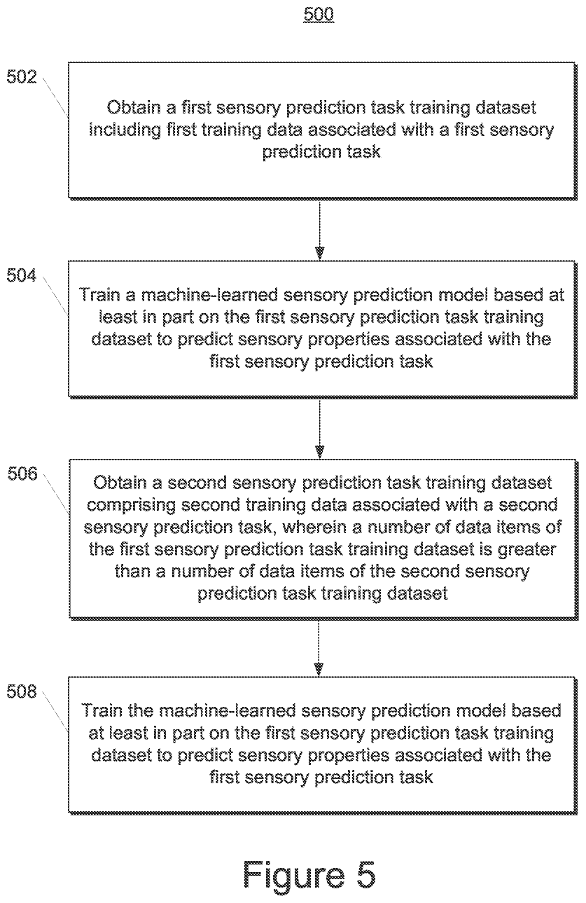

According to example aspects of the present disclosure, the machine-learned sensory prediction model can be trained using a first sensory prediction task training dataset for a first sensory prediction task. In some implementations, the model can further be trained on a second sensory prediction task training dataset for a second sensory prediction task. In some implementations, a number of data items of the first sensory prediction task training dataset can be greater than a number of data items of the second sensory prediction task training dataset. For instance, in some implementations, the machine-learned sensory prediction model can be trained according to the method 500 of FIG. 5. Furthermore, in some implementations, the model may be trained only for the first sensory prediction task, such as if no training data is available for the second sensory prediction task. The model may nonetheless be useful for the second sensory prediction task.

In some implementations (e.g., for the first prediction task), the machine-learned model can be trained using training data that includes descriptions of molecules (e.g., graphical descriptions of chemical structures of molecules) that have been labeled (e.g., manually by an expert) with descriptions of sensory properties (e.g., olfactory properties) (e.g., textual descriptions of odor categories such as “sweet,” “piney,” “pear,” “rotten,” etc.) that have been assessed for the molecules. The trained machine-learned prediction model can provide prediction data that predicts the smell of previously unassessed molecules.

More particularly, most machine learning models require regularly-shaped input (e.g. a grid of pixels, or a vector of numbers) as input. However, GNNs enable the use of irregularly-shaped inputs, such as graphs, to be used directly in machine learning applications. As such, according to an aspect of the present disclosure, by viewing atoms as nodes, and bonds as edges, a molecule can be interpreted as a graph. Example GNNs are learnable permutation-invariant transformations on nodes and edges, which produce fixed-length vectors that are further processed by a fully-connected neural network. GNNs can be considered learnable featurizers specialized to a task, in contrast with expert-crafted general features.

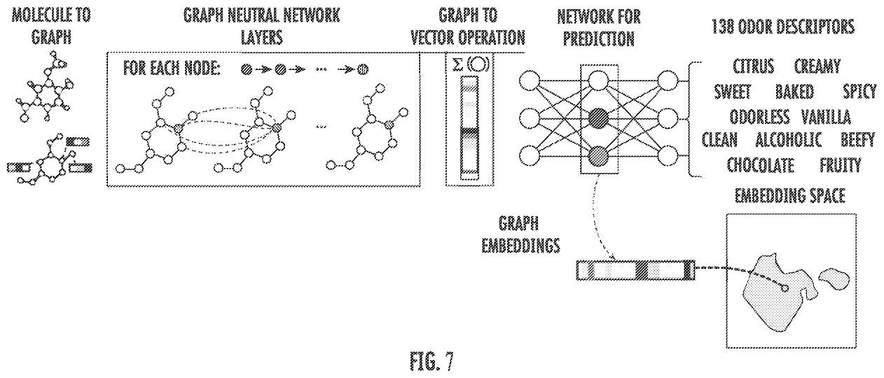

Some example GNNs include one or more message passing layers, each followed by a reduce-sum operation, followed by several fully connected layers. The example final fully-connected layer has a number of outputs equal to the number of odor descriptors being predicted. One example model is illustrated in FIG. 7, which illustrates an example model schematic and data flow. In the example illustrated in FIG. 7, each molecule is first featurized by its constituent atoms, bonds, and connectivities. Each Graph Neural Network (GNN) layer transforms the features from the previous layer. The output from the final GNN layer is reduced to a vector, which is then used for predicting odor descriptors via a fully-connected neural network. In some example implementations, graph embeddings can be retrieved from the penultimate layer of the model.

Referring again to FIG. 4, at 404, method 400 can include obtaining, by the one or more computing devices, input data (e.g., a graph) that (e.g., graphically) describes a chemical structure of a selected molecule. For instance, an input graph structure of a molecule's chemical structure (e.g., a previously unassessed molecule, etc.) can be obtained for use in predicting one or more perceptual (e.g., olfactory) properties of the molecule. For example, in some embodiments, a graph structure can be obtained based on a standardized description of a molecule's chemical structure, such as a simplified molecular-input line-entry system (SMILES) string, and/or the like. In some embodiments, in response to receipt of a SMILES string or other description of chemical structure, the one or more computing devices can convert the string to a graph structure that graphically describes the two-dimensional structure of a molecule. Additionally or alternatively, the one or more computing devices could provide for creating a three-dimensional representation of the molecule, for example using quantum chemical calculations, for input to a machine-learned model.

At 406, method 400 can include providing, by the one or more computing devices, the input data that describes the chemical structure of the selected molecule as input to the machine-learned graph neural network. For example, the graph structure descriptive of a molecule's chemical structure, obtained at 404, can be provided to a machine-learned model (e.g., a trained graph convolutional neural network and/or other type of machine-learned model) that can predict, from either the graph structure or features derived from the graph structure, sensory properties (e.g., olfactory properties) of the molecule.

At 408, method 400 can include receiving, by the one or more computing devices, prediction data descriptive of one or more predicted sensory properties (e.g., olfactory properties) of the selected molecule as an output of the machine-learned graph neural network. In particular, the machine-learned model may provide output prediction data comprising a description of predicted perceptual properties of the molecule, such as, for example, a list of olfactory perceptual properties descriptive of what the molecule would smell like to a human. For instance, a SMILES string can be provided, such as the SMILES string “O=C(OCCC(C)C)C” for the chemical structure of isoamyl acetate, and the machine-learned model can provide as output a description of what that molecule would smell like to a human, for example, a description of the molecule's odor properties such as “fruit, banana, apple”.

In some example embodiments, the prediction data can indicate whether or not the molecule has a particular desired olfactory perceptual quality (e.g., a target scent perception, etc.). In some example embodiments, the prediction data can include one or more types of information associated with a predicted sensory property (e.g., olfactory property) of a molecule. For instance, prediction data for a molecule can provide for classifying the molecule into one sensory property (e.g., olfactory property) class and/or into multiple sensory property (e.g., olfactory property) classes. In some instances, the classes can include human-provided (e.g., experts) textual labels (e.g., sour, cherry, piney, etc.). In some instances, the classes can include non-textual representations of scent/odor, such as a location on a scent continuum or the like. In some example embodiments, prediction data for molecules can include intensity values that describe the intensity of the predicted scent/odor. In some example embodiments, prediction data can include confidence values associated with the predicted olfactory perceptual property. In some example embodiments, in addition or alternatively to specific classifications for a molecule, prediction data can include numerical embedding that allows for similar search or other comparisons between two molecules based on a measure of distance between two embeddings.

At 410, method 400 can include providing, by the one or more computing devices, the prediction data descriptive of the one or more predicted sensory properties (e.g., olfactory properties) of the selected molecule as an output.

In some implementations, the method 400 can further include obtaining, by the one or more computing devices, a second graph that graphically describes a second chemical structure of a second selected molecule, providing, by the one or more computing devices, the second graph that graphically describes the second chemical structure of the second selected molecule as input to the machine-learned graph neural network, receiving, by the one or more computing devices, second prediction data descriptive of one or more second sensory properties associated with the second selected molecule as an output of the machine-learned graph neural network, and determining, by the one or more computing devices, one or more sensory differences between the selected molecule and the second selected molecule based on a comparison of the prediction data for the selected molecule with the second prediction data for the second selected molecule. For instance, this can provide that a plurality of molecules can be compared to determine which of the molecules exhibits desirable sensory qualities.

FIG. 5 depicts a flowchart diagram of an example method 500 for training a sensory prediction model for predicting sensory properties for a prediction task having limited available training data according to example embodiments of the present disclosure. Although FIG. 5 depicts steps performed in a particular order for purposes of illustration and discussion, the methods of the present disclosure are not limited to the particularly illustrated order or arrangement. The various steps of the method 500 can be omitted, rearranged, combined, and/or adapted in various ways without deviating from the scope of the present disclosure. Method 500 can be implemented by one or more computing devices, such as one or more of the computing devices depicted in FIGS. 1A-1C.

At 502, method 500 can include obtaining, by a computing system comprising one or more computing devices, a first sensory prediction task training dataset comprising first training data associated with a first sensory prediction task. In some implementations, the first prediction task can be associated with a first species, such as humans. For instance, the first prediction task training dataset can include perception data associated with the first species, such as human perception data.

At 504, method 500 can include training, by the computing system, a machine-learned sensory prediction model based at least in part on the first sensory prediction task training dataset to predict sensory properties associated with the first sensory prediction task. For instance, in some implementations, the machine-learned sensory prediction model can include a sensory embedding model. Training the machine-learned sensory prediction model based at least in part on the first sensory prediction task training dataset can include training the sensory embedding model with a first prediction task model based at least in part on the first sensory prediction task training dataset

At 506, method 500 can include obtaining, by the computing system, a second sensory prediction task training dataset comprising second training data associated with a second sensory prediction task. A number of data items of the first sensory prediction task training dataset can be greater than a number of data items of the second sensory prediction task training dataset. In some implementations, the second prediction task can be associated with a second species, where the second species is different from the first species. For instance, the second sensory prediction task training dataset can include sensory perception data associated with the second species, such as nonhuman perception data.

At 508, method 500 can include training, by the computing system, the machine-learned sensory prediction model based at least in part on the second sensory prediction task training dataset to predict sensory properties associated with the second sensory prediction task. Training the machine-learned sensory prediction model based at least in part on the second sensory prediction task training dataset can include training the sensory embedding model with a second prediction task model based at least in part on the second sensory prediction task training dataset. It should be understood that in some embodiments, the model may be trained with only a first prediction task dataset and used to output predictions for a second prediction task.

In some implementations, the sensory embedding model is configured to produce a sensory embedding and wherein the first sensory prediction task model and the second sensory prediction task model are configured to receive the sensory embedding as input. In some implementations, at least one of the first training data or the second training data includes a plurality of example chemical structures, where each example chemical structure is labeled with one or more sensory property labels that describe sensory properties of the example chemical structure.

Thus, a machine-learned sensory prediction model may be trained on a first sensory prediction task and used to output predictions associated with a second sensory prediction task. As one example, the first sensory prediction task may be a broader sensory prediction task than the second sensory prediction task. For example, the model may be trained on a broad task and transferred to a narrow task. As one example, the first task may be a broad property task and the second task may be a specific property task (e.g., olfactory). Additionally and/or alternatively, the first sensory prediction task may be a task for which a larger amount of training data is available than for the second sensory prediction task. Additionally and/or alternatively, the first sensory prediction task may be associated with a first species and the second sensory prediction task may be associated with a second species. As one example, the first sensory prediction task may be a human olfactory task. Additionally and/or alternatively, the second sensory prediction task may be a pest control task, such as a mosquito repellent task.

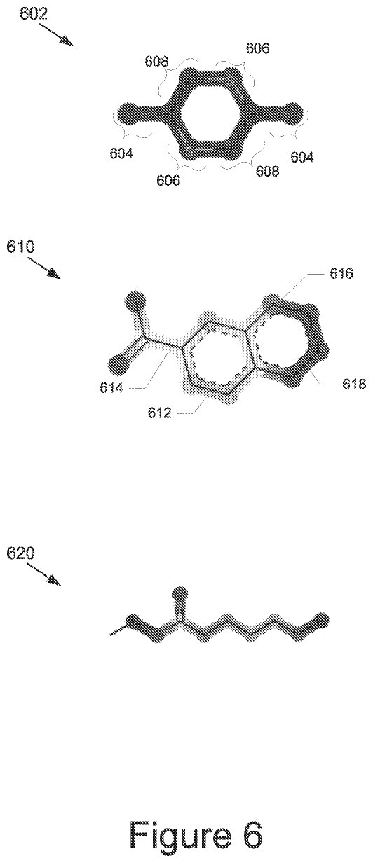

FIG. 6 depicts example illustrations for visualizing structural contribution associated with predicted sensory properties (e.g., olfactory properties), such as for the second sensory prediction task, according to example embodiments of the present disclosure. As illustrated in FIG. 6, in some embodiments, the systems and methods of the present disclosure can provide output data to facilitate interpreting and/or visualizing which aspects of a molecule's structure most contributes to its predicted sensory quality. For example, in some embodiments, a heat map could be generated to overlay the molecule structure, such as visualizations 602, 610, and 620, that provides indications of which portions of a molecule's structure are most important to the perceptual properties of the molecule and/or which portions of a molecule's structure are less important to the perceptual properties of the molecule. As an example, a heat map visualization, such as visualization 602, may provide indications that atoms/bonds 604 may be most important to the predicted perceptual properties, that atoms/bonds 606 may be moderately important to the predicted perceptual properties, and that atoms/bonds 608 may be less important to the predicted perceptual properties. In another example, visualization 610 may provide indications that atoms/bonds 612 may be most important to the predicted perceptual properties, that atoms/bonds 614 may be moderately important to the predicted perceptual properties, and that atoms/bonds 616 and atoms/bonds 618 may be less important to the predicted perceptual properties. In some implementations, data indicative of how changes to a molecule structure would impact sensory (e.g., olfactory) perception can be used to generate visualizations of how the structure contributes to a predicted sensory (e.g., olfactory) quality. For example, iterative changes to the molecule's structure (e.g., a knock-down technique, etc.) and their corresponding outcomes can be used to evaluate which portions of the chemical structure are most contributory to the sensory (e.g., olfactory) perception.

Some example neural network architectures described herein can be configured to build representations of input data at their intermediate layers. The success of deep neural networks in prediction tasks relies on the quality of their learned representations, often referred to as embeddings. The structure of a learned embedding can even lead to insights on the task or problem area, and the embedding can even be an object of study itself. According to example aspects of the present disclosure, these embeddings can even be used to transfer information learned for a first sensory prediction task for use with a second sensory prediction task which may have limited training data, such that it would be difficult or impossible to model the second sensory prediction task otherwise.

Some example computing systems can save the activations of the penultimate fully connected layer as a fixed-dimension “sensory embedding”. The GNN model can transform a molecule's graph structure into a fixed-length representation that is useful for classification. A learned GNN embedding on an odor prediction task may include a semantically meaningful and useful organization of sensory properties of molecules.

A sensory embedding representation that reflects common-sense relationships between odors should show structure both globally and locally. Specifically, for global structure, sensory properties that are perceptually similar should be nearby in a sensory embedding representation. For local structure, individual molecules that have similar sensory percepts should cluster together and thus be nearby in the embedding.

Example sensory embedding representations of each data point can be produced from the penultimate-layer output of an example trained GNN model. For example, each molecule can be mapped to a 63-dimensional vector. Qualitatively, to visualize this space in 2D, principal component analysis (PCA) can optionally be used to reduce its dimensionality. The distribution of all molecules sharing a similar label can be highlighted using kernel density estimation (KDE).

One example global structure of the embedding space is illustrated in FIG. 8. In this example, we find that individual odor or olfactory descriptors (e.g. musk, cabbage, lily and grape) tend to cluster in their own specific region. For odor descriptors that co-occur frequently, we find that the embedding space captures a hierarchical structure that is implicit in the odor descriptors. The clusters for odor labels jasmine, lavender and muguet are found inside the cluster for the broader odor label floral. This example embedding space is illustrated with respect to, for example, a human olfactory perception space. According to example aspects of the present disclosure, these embeddings may additionally be utilized in a second sensory perception task space, such as a pest repellent space.

FIGS. 8A and 8B illustrate a 2D representation of a GNN model embeddings as a learned odor space. Molecules are represented as individual points. Shaded and contoured areas are kernel density estimates of the distribution of labeled data. A. Four odor descriptors with low co-occurrence have low overlap in the embedding space. B. Three general odor descriptors (floral, meaty, alcoholic) each largely subsume more specific labels within their boundaries. Example experiments have indicated that the generated embeddings can be used to retrieve molecules that are perceptually similar to a source molecule (e.g., using a nearest neighbor search over the embeddings).

The technology discussed herein makes reference to servers, databases, software applications, and other computer-based systems, as well as actions taken and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure cover such alterations, variations, and equivalents.

What is claimed is:

1. A computer-implemented method for training a sensory prediction model for predicting sensory properties for a prediction task having limited available training data for a second sensory prediction task, the computer-implemented method comprising:
obtaining, by a computing system comprising one or more computing devices, a first sensory prediction task training dataset comprising first training data associated with a first sensory prediction task, the first training data comprising molecular structure data labeled with first sensory properties associated with the first sensory prediction task;
training, by the computing system, a machine-learned sensory prediction model based at least in part on the first sensory prediction task training dataset to predict the first sensory properties associated with the first sensory prediction task;
obtaining, by the computing system, a second sensory prediction task training dataset comprising second training data associated with a second sensory prediction task, the second training data comprising molecular structure data labeled with second sensory properties associated with the second sensory prediction task, wherein a number of data items of the first sensory prediction task training dataset is greater than a number of data items of the second sensory prediction task training dataset; and
training, by the computing system, the machine-learned sensory prediction model based at least in part on the second sensory prediction task training dataset to predict the second sensory properties associated with the second sensory prediction task.

2. The method of claim 1, wherein the machine-learned sensory prediction model comprises a sensory embedding model, wherein training the machine-learned sensory prediction model based at least in part on the first sensory prediction task training dataset comprises training the sensory embedding model with a first prediction task model based at least in part on the first sensory prediction task training dataset, and wherein training the machine-learned sensory prediction model based at least in part on the second sensory prediction task training dataset comprises training the sensory embedding model with a second prediction task model based at least in part on the second sensory prediction task training dataset.

3. The method of claim 2, wherein the sensory embedding model is configured to produce a sensory embedding and wherein the first sensory prediction task model and the second sensory prediction task model are configured to receive the sensory embedding as input.

4. The method of any preceding claim, wherein at least one of the first training data or the second training data comprises a plurality of example chemical structures, each example chemical structure labeled with one or more sensory property labels that describe sensory properties of the example chemical structure.

5. The method of any preceding claim, wherein the first prediction task is associated with a first species and wherein the second prediction task is associated with a second species, the second species being different from the first species.

6. The method of any preceding claim, wherein the first sensory prediction task training dataset comprises human perception data and the second sensory prediction task training dataset comprises nonhuman perception data.

7. A computer-implemented method for predicting sensory properties for a prediction task having limited available training data, the computer-implemented method comprising:
obtaining, by one or more computing devices, a machine-learned sensory prediction model trained to predict sensory properties of molecules based at least in part on chemical structure data associated with the molecules, wherein the machine-learned sensory prediction model is trained using a first sensory prediction task training dataset for a first sensory prediction task;
obtaining, by the one or more computing devices, input data that describes a chemical structure of a selected molecule;
providing, by the one or more computing devices, the input data that describes the chemical structure of the selected molecule as input to the machine-learned sensory prediction model;
receiving, by the one or more computing devices, prediction data descriptive of one or more second sensory properties of the selected molecule associated with a second sensory prediction task as an output of the machine-learned sensory prediction model; and
providing, by the one or more computing devices, the prediction data descriptive of the one or more second sensory properties of the selected molecule as an output.

8. The computer-implemented method of claim 7, wherein the sensory prediction model is further trained using a second sensory prediction task training dataset for the second sensory prediction task, wherein a number of data items of the first sensory prediction task training dataset is greater than a number of data items of the second sensory prediction task training dataset.

9. The computer-implemented method of claim 7 or 8, wherein the one or more second sensory properties associated with the second sensory prediction task comprise one or more of:
optical properties of the selected molecule;
gustatory properties of the selected molecule;
biodegradability of the selected molecule;
stability of the selected molecule; or
toxicity of the selected molecule.

10. The computer-implemented method of any of claims 7 through 9, wherein the sensory prediction model comprises one or more graph neural networks, and wherein the input data comprises a graph that graphically describes a chemical structure of a selected molecule.

11. The computer-implemented method of claim 10, wherein the graph that graphically describes the chemical structure of the selected molecule comprises a two-dimensional graph structure indicative of a two-dimensional representation of the chemical structure of the selected molecule.

12. The computer-implemented method of claim 10, wherein the graph that graphically describes the chemical structure of the selected molecule comprises a three-dimensional graph structure indicative of a three-dimensional representation of the chemical structure of the selected molecule, and wherein the method further comprises performing, by the one or more computing devices, one or more quantum chemical calculations to identify the three-dimensional representation of the chemical structure of the selected molecule.

13. The computer-implemented method of any of claims 7 through 12, further comprising:
performing, by the one or more computing devices, an iterative search process to identify an additional molecule that exhibits one or more desired sensory properties associated with the second prediction task, wherein the iterative search process comprises, for each of a plurality of iterations:
generating, by the one or more computing devices, a candidate molecule graph that graphically describes a candidate chemical structure of a candidate molecule;
providing, by the one or more computing devices, the candidate molecule graph that graphically describes the candidate chemical structure of the candidate molecule as input to the machine-learned graph neural network;
receiving, by the one or more computing devices, prediction data descriptive of one or more predicted sensory properties of the candidate molecule as an output of the machine-learned graph neural network; and
comparing, by the one or more computing devices, the one or more predicted sensory properties of the candidate molecule to the one or more desired sensory properties.

14. The method of any of claims 7 through 13, wherein the prediction data indicative of the one or more predicted sensory properties of the selected molecule comprises a numerical embedding; and
the method further comprises identifying, by the one or more computing devices, other molecules that have sensory properties that are similar to the predicted sensory properties of the selected molecule by comparing the numerical embedding with other numerical embeddings output for the other molecules by the machine-learned graph neural network.

15. The computer-implemented method of any of claims 7 through 14, further comprising:
generating, by the one or more computing devices, visualization data descriptive of a relative importance of one or more structural units of chemical structure of the selected molecule to the predicted sensory properties associated with the selected molecule and the second prediction task; and
providing, by the one or more computing devices, the visualization data in association with the prediction data indicative of the one or more olfactory properties.

16. The computer-implemented method of any of claims 7 through 15, further comprising:
generating, by the one or more computing devices, data indicative of how a structural change to the chemical structure of the selected molecule affects the predicted sensory properties associated with the selected molecule.

17. The method of any preceding claim, wherein the first prediction task is associated with a first species and wherein the second prediction task is associated with a second species, the second species being different from the first species.

18. One or more non-transitory computer-readable media comprising a sensory embedding, the sensory embedding generated as output from a machine-learned embedding model, wherein the machine-learned embedding model is trained using a first sensory prediction task training dataset for a first sensory prediction task and a second sensory prediction task training dataset for a second sensory prediction task, wherein a number of data items of the first sensory prediction task training dataset is greater than a number of data items of the second sensory prediction task training dataset.

19. A composition of matter having a molecular structure designed based at least in part on a sensory embedding to exhibit one or more desired sensory properties, the sensory embedding generated as output from a machine-learned embedding model in response to receipt of input data descriptive of the molecular structure, wherein the machine-learned embedding model is trained using a first sensory prediction task training dataset for a first sensory prediction task and the embedding is used for a second sensory prediction task.

20. A method of using the composition of matter of claim 19, comprising applying the composition of matter to a region such that the region exhibits the one or more desired sensory properties.
